(19) United States
(12) Patent Application Publication
(10) Pub. No.: US 2010/0015226 A1
(43) Pub. Date: Jan. 21, 2010

(54) BIORESORBABLE HYDROGEL

(75) Inventors: Antoine Turzi, Mollens (CH); Guy Fortier, Montreal (CA); Yaode Liu, Montreal (CA)

Correspondence Address: SALIWANCHIK LLOYD & SALIWANCHIK;A PROFESSIONAL ASSOCIATION, PO Box 142950, GAINESVILLE, FL, 32614, US

(73) Assignee: Antoine Turzi, Mollens (CH)

(21) Appl. No.: 11/917,967

(22) PCT Filed: Jun. 21, 2005
(86) PCT No.: PCT/IB2005/001742

§ 371 Date: Dec. 18, 2007

(51) Int. Cl. A61K 9/10; A61K 38/38

(52) U.S. Cl. 424/484; 514/12

(57) ABSTRACT

The invention relates to a water-swellable, hydrophilic, physically-crosslinked hydrogel which can slowly disintegrate in an aqueous medium. The inventive hydrogel essentially consists of albumin blood proteins which are bound in the form of a gel in a basic medium. The invention also relates to the use of said hydrogel as a bioresorbable separation membrane and to a method of preparing same.

The present invention relates to a hydrophilic gel which can disintegrate in an aqueous medium, a method of preparing same, as well as a bioresorbable separation membrane that can be used in particular for surgery and more particularly with the aim of minimising and/or preventing the formation of post-surgical adherences.

The formation of post-operative adherences is a frequent occurrence during cardiac, abdominal and pelvic surgery procedures. These adherences can degenerate into obstructive pathologies depending on the extent and severity thereof. In the case of abdominal surgery, the formation of adhesions can, for example, compromise organ mobility and lead to chronic pains in the patient. Adhesions are responsible for 49 to 74% of small-intestine obstructions, 10 to 15% of cases of infertility, and 20 to 50% of chronic pelvic pain in women. Furthermore, during re-operations, the presence of adhesions can significantly increase the complexity and duration of the surgery, as well as post-surgical complications. The same conclusions also apply in the case of cardiac, pulmonary and thoracic surgery. The presence of adhesions is an influencing factor both on surgical risk (mortality and morbidity) and the cost of treatment.

The formation of adherences occurs during the natural repair process of healthy tissue and consist of fibrous adhesions between organs undergoing surgery or between a separation membrane such as the peritoneum or the pericardium and the subjacent organs. By way of example, if the peritoneum is damaged during surgery, fibrin builds up on its surface. These strips of fibrin act like glue, causing the organ and membrane to bond together. In ideal circumstances, following repair to the site, the fibrin at the site of the mesothelial damage would be destroyed by the plasmin. The plasmin is formed from the action of the tissue plasminogen activator (mesothelial cells) on the blood plasminogen. Fibrinolysis and the coagulation cascade are involved in this pathology. Fibroblast recruitment and proliferation as well as the presence of inflammatory cells are also observed, at the same time as collagen deposition. An endothelial cell-controlled angiogenesis mechanism may possibly play a role in the formation of adhesions.

One of the innovative strategies currently put forward to limit the formation of adhesions, without interfering with the natural repair mechanism, consists in inserting a dried or un-dried sheet of synthetic or natural polymer, which may or may not be chemically cross-linked, between the damaged membrane and the subjacent organs. The dried sheet is hydrated in contact with the tissues and is eroded by a slow dissolution or by hydrolysis. This process leads to the sheet being fully resorbed during the weeks following implantation. The formation of adhesions that starts as soon as the operation is over comes to an end approximately 7 days later. Therefore, if, during the first week, a physical barrier is placed for example between the pericardium and the epicardium, or between the peritoneum and the organs of the gastrointestinal system, or between the pleura and the lung, the number of adhesion sites in these areas should be kept to a minimum. When the barrier has disappeared, adhesions that are attached at one end only, should be observed. This barrier or separator should have biodegradable, malleable, biocompatible, non-abrasive and easy to synthesise properties.

The term hydrogel refers by definition to a natural or artificial polymeric material that can hold several times its own weight in water, thereby putting it on a par with living tissue in terms of hydration. Therefore, it appears that hydrogels can act as a barrier, i.e. separation sheets.

The majority of hydrated polymeric films are obtained by chemically crosslinking natural or synthetic hydrophilic polymers in the presence of bifunctional agents or by monomer polymerisation, which gives them high structural stability and as a result low biodegradability. Such films are useful in external applications such as a sticking plaster and for controlled release of medications. They have limited in vivo uses and applications as they decompose very slowly, when they actually do decompose. There exists, therefore, a requirement in the field of medicine for biodegradable, hydrated hydrogel films that could be used in vivo during surgery to reduce the occurrence of adhesions and even allow controlled in situ release of medication. Several types of dry polymeric films exist for the purpose of providing controlled release of medication and which break down through dissolution. These dry films cannot be implanted in certain areas of the human body as they are solid, inflexible and abrasive, and require to be implanted in a location offering no mechanical restraint, unlike a hydrated hydrogel which by its nature is flexible and non-abrasive. The biodegradable, hydrated hydrogel film can be placed in any part of the body, including those subject to mechanical stress such as joints, the heart and the peritoneal cavity. Once it has decomposed, not a single trace of the hydrogel should be found at the site of administration, thereby removing any risk regarding polymer bioaccumulation.

The majority of hydrated polymeric films are obtained by chemically crosslinking natural or synthetic hydrophilic polymers in the presence of bifunctional agents or by monomer polymerisation, which gives them high structural stability and as a result low biodegradability. Therefore, for in vivo use, the hydrogel films must break down within a sufficiently short space of time, in other words in less time than it takes for a fibrous capsule to form as the result of inflammatory cell response around the implant in the host. The timescale for breakdown here is from 20 days to a few weeks.

Irradiated human amniotic membranes (Young et al., Fertil. Steril., 55:624-628, 1991) have been used with a certain degree of success in the rabbit model during pelvic surgery. However, ethical problems and supply issues have restricted its development. Two hydrogels have been assessed in the rat model (West and Hubbell, Biomaterials, 16: 1153-1156, 1995), the first is an in situ photopolymerised polyethylene glycol-co-lactic acid diacrylate hydrogel and the second a physically crosslinked polyethylene glycol-co-polypropylene glycol hydrogel, Poloxamer 407. Reduced adherence of 75 and 38% respectively was observed; the respective biodegradation times were 4 days for the first and 2 days for the second. More recently, a hydrogel barrier obtained from two liquids containing polyethylene glycol sprayed on to the site of pelvic surgery in a porcine model led to a significant reduction of 60% in the number and severity of adhesions. The hydrogel layer was absorbed in 5 days (Ferland et al, Hum. Reprod., 16 : 2718.2723, 2001).

Crosslinked hyaluronic acid hydrogels have resulted in a significant reduction of abdominal adhesion formation and reformation in rabbits, but the degradability property of the polymer was not discussed (Osada et al., J. Int. Med. Res., 27:233-241, 1999). During one clinical study, membranes made of hyaluronic acid and carboxymethylcellulose led to a 51% reduction in cases of adhesion and a 87% reduction in their severity (Becker et al., J. Am. Coll. Surg., 183 : 297-306, 1996) following abdominal surgery.

In the case of heart surgery, the first membranes for limiting the formation of epicardium-pericardium adhesions were made of poly(2-hydroxyethyl methacrylate) reinforced with poly(ethylene terephthalate) (Walker et al., Asaio J. 38:M550-554, 1992). These fairly rigid hydrogel membranes were not biodegradable and were prone to calcification after 9 months implantation in dogs. Poly(hydroxybutyrate) membranes offered good results in sheep, but were only resorbed very slowly by macrophages after more than 30 months (Malm et al., J. Thorac. Cardiovasc. Surg. 104:600-607, 1992). Similar experiments in dogs using resorbable membranes made of polyethylene glycol and polylactic acid showed a reduction in adhesion formation, yet biodegradability was very quick and the resorption was complete after a few days (Okuyama et al., Ann. Thorac. Surg. 68:913-918, 1999). Resorbable collagen-elastin membranes (Florez et al., J. Thorac. Cardiovasc. Surg., 117:185, 1999) have been used to successfully reduce the number of adhesions forming during heart surgery in humans. More recently, N-O carboxymethylchitosan membranes have been used to the same ends, this time in rabbits, and have led to a very significant reduction in the number of adhesions, but the biodegradability of the sheets was not discussed (Krause et al., J. Invest. Surg., 14:93-97, 2001).

The aim of said invention is to provide a new type of hydrogel intended to form a biodegradable separation membrane, designed to overcome the drawbacks of known polymeric films, and which offers excellent biocompatibility, good in vivo degradability, viscoelastic properties that ensure easy handling and an absence of abrasive action.

A first purpose of this invention intended to reach the aforementioned goal is therefore a water-swellable, hydrophilic, physically crosslinked hydrogel which can slowly disintegrate in an aqueous medium, and which essentially consists of albumin blood proteins that are bound in the form of a gel in a basic medium.

A second purpose of the invention consists of a bioresorbable separation membrane formed of a film of the aforementioned hydrogel.

A third purpose of the invention is a process for the preparation of said hydrogel, which consists of mixing an aqueous solution of protein albumin with a base, and then leaving the mixture to rest until polymerisation of the albumin macromolecules.

Finally, the purpose of the invention is also to use the separation membrane in veterinary and human surgery, as well as for the controlled administration of a therapeutically active substance. The albumin blood proteins can be isolated from the plasma or serums of animal (bovine, rabbit, porcine, etc) or human origin.

Human albumin may be obtained from a blood bank or by genetic engineering, or may be of an autologous nature. In the latter event, it appears that patient acceptability of the implant will be markedly higher given the autologous composition of the membrane.

With regard to the preparation procedure of the hydrogel according to the invention, it is preferably characterised in that the aqueous albumin solution is comprised between 10 and 20% (weight/vol.), that the base is NaOH 5N and that the ratio between the NaOH and the albumin solution is between 0.5 and 2.0% (weight/vol.), and by the fact that the polymerisation lasts between 1 hour and 8 hours. It is also preferably characterised in that all the steps in the procedure according to the invention are carried out under sterile conditions.

As regards the biodegradable separation membrane according to the invention, it can be advantageously used by a surgeon or veterinarian to reduce the formation of adhesions during thoracic, pelvic, abdominal, cardiac and other forms of surgery.

The hydrogel forming the biodegradable separation membrane can also contain a therapeutically active substance, or a system of controlled release of medication with a physiologically acceptable form, which is suitable for administration by oral, rectal, vaginal route or in the form of a surgical implant.

Finally, the separation membrane can also be altered at the surface by the covalent addition of monoethoxy polyethylene-glycol chains of varying molecular masses or even by incubation in a polyethylene-glycol solution in order to alter its biocompatibility and biodegradability properties.

Accordingly, the novel hydrogel according to the invention possesses particularly interesting biomedical properties, such as its ability to have the form of a thin film of adjustable size, its very high water content, mechanical properties that enable easy handling, the fact that it is translucent thereby facilitating positioning, and its biodegradable property in situ in a physiological environment.

The said invention will now be illustrated using the following examples:

### EXAMPLE 1

### Synthesis of the Resorbable Serum Albumin Hydrogel

By way of example, a commercially available serum albumin such as but not restricted to rabbit or bovine serum albumin, when dissolved in distilled water to a concentration varying between 10 and 20% (weight/volume) and a base, for example an aqueous solution of 5.0 N NaOH is added to give a final base percentage of between 1.5 and 0.6% (w/v); this albumin will form a gel after sitting at room temperature for a period that can range from 1 hour to 8 hours. This gelling of the albumin provides a gel that is hydrated, malleable, translucent and which proves to be a hydrogel with a water content of up to 97% (see Table 1). The texture of the hydrogels obtained by varying the amount of base and albumin ranges from very viscoelastic to vitreous, whilst remaining flexible and hydrated. The pH of the hydrogel can be adjusted to the physiological pH or to another pH level simply by being incubated for 1 hour in the desired physiological solution. During this incubation and balancing period, the saline buffer solution will be replaced twice. To obtain a hydrogel of the desired size and thickness, the albumin solution that has had the required amount of base solution freshly added can be poured between two plates of clean glass which are separated by spacer bars of the desired thickness (0.75 to 2.0 mm) and which further provide the assembly with the required watertight properties. An assembly of this type used to prepare polyacrylamide gels for electrophoresis can be useful for this purpose.

TABLE 1
Conditions of synthesis of the serum albumin hydrogel
and properties of the resulting hydrogels.

| | | | | | Weight after | |
|---|---|---|---|---|---|---|
| Quantity¹ AS | NaOH² 5.0N | Ratio | Gelling | | stripping⁴ | EWC⁵ |
| % (w/v) | % (w/v) (ul) | % AS/% Base | + or − | Hardness³ | g | % |
| 10 | 2.4 (120) | 4.2 | − | − | n.a. | n.a. |
| 10 | 1.2 (60) | 8.4 | ± | ± | 3.04 | 96 |
| 10 | 1.0 (50) | 10.0 | + | + | 3.72 | 97 |
| 10 | 0.8 (40) | 12.5 | + | ++ | 3.43 | 97 |
| 10 | 0.6 (30) | 16.7 | + | +++ | 3.40 | 97 |
| 17.5 | 1.2 (60) | 14.6 | + | ++++ | 2.8 | 93 |
| 17.5 | 0.6 (30) | 29.2 | + | ++++ | 4.37 | 96 |
| 20 | 1.2 (60) | 16.7 | + | ++++ | n.d. | n.d. |
| 20 | 0.6 (30) | 33.3 | + | ++++ | n.d. | n.d. |

¹initial bovine serum albumin solution (100 mg/ml water (10% w/v) etc., 1 ml was used for the hydrogel synthesis.
²final % (w/v) of NaOH in the albumin solution, the volume in micro litres of the aqueous solution 5N NaOH added to 1 ml of the albumin solution is shown in brackets.
³hardness of the hydrogel evaluated manually; ± viscous gel, difficult to handle, + very viscoelastic gel, ++ slightly vitreous, +++ vitreous and ++++ very vitreous, easily handled, crumbly when pressed between the fingers.
⁴weight of the hydrogel after being rinsed for 1 hour in distilled water containing 0.02% sodium azide as a preservative.
⁵EWC, % equilibrated water of the hydrogel = ((wet weight − theoretical dry weight)/wet weight) × 100. The theoretical dry weight equates to the amounts of albumin and NaOH used to synthesise the hydrogel.

### EXAMPLE 2

### Autologous Human or Animal Hydrogel

Following the previous method, the animal albumin is replaced by human albumin isolated from the blood of the patient or of the animal that is destined to receive the hydrogel by surgical means. The volume of blood taken will be a function of the size and number of hydrogels to be synthesised. To do this, the albumin is isolated from a volume of blood that has been freshly taken using the Hao method (Hao, Vox. Sang. 36:313-320, 1979), which is given here for example purposes only. All the steps are performed at a temperature of between −5 and 0° C. In short, this method consists of diluting a volume of blood plasma by two volumes of an aqueous solution of 0.15 M NaCI, and the pH is adjusted to 5.6 by the addition of a quantity of a solution of sodium acetate 0.8 M at pH 4.0. Once the mixture has cooled, an aqueous ethanol solution of 95% v/v is slowly added while being stirred until a final ethanol concentration of 42% (v/v) is obtained. Stirring is then continued for one hour and the solution is centrifuged at 12,000 g/lh. The pH of the supernatant is then adjusted to 4.8 using the acetate buffer solution. The solution is then stirred for 1 h and left unstirred for 3 h. The solution is then centrifuged at 12,000 g/lh. The precipitate is then collected and contains the albumin. The albumin is dissolved in water and the amount of protein is assayed by the bicinchoninic acid method (BCA, Pierce, USA). The albumin is diluted using sterile water to give the desired final concentration (between 15 and 20% w/v), and then filtered over a membrane porous to 0.01 micron in order to be sterilised.

The hydrogel is obtained according to the method described previously. Furthermore, all handling manoeuvres are carried out in such ways that the hydrogel is sterile.

### EXAMPLE 3

### Implant of a Hydrogel in the Pericardium-Epicardium Position in Rabbits to Prevent Post-Surgical Adhesions

In order to assess the protection offered by the hydrogel against adherences arising from heart surgery and to assess the in vivo resorption speed of the hydrogel, the hydrogels will be implanted in the heart cavity of rabbits.

To achieve this, hydrogels measuring 25×20×1 mm are prepared by mixing 150 mg of rabbit albumin (Sigma, USA) per ml of 0.9% NaCl solution to which is added 50 ml of 5.0N NaOH and a drop of methylene blue 1% to colour the hydrogel. An Ethicon 910 suture (J&J, USA) was added beforehand between the glass sheets to allow the surgeon to secure the hydrogel to the inside of the pericardium. After synthesis, the hydrogels are washed several times and preserved in 0.9% NaCl. All the solutions are filtered over a 0.2 micron Millipore filter and all handling manoeuvres carried out in sterile conditions.

The rabbits are anaesthetized with isoflurane (1.5%) after receiving a premedication of 0.5 mg atropine, 0.5 mg/kg midazolam and 6 mg/kg azaperone. After the muscles have been relaxed with 0.2 mg/kg pancuronium administered via the right jugular, the animals are intubated and mechanically ventilated. A 10 mg/kg dose of fentanyl is administered at the start of the surgery, then repeated with a 0.05 mg dose every 30 minutes. The rabbit is laid out in the dorsal decubitus position. After the thorax is shaved and disinfected, a median sternotomy is performed and the various planes dissected in order to land on the pericardium which is opened. The pericardium is closed immediately with a continuous suture using Vicryl/0 (rabbit checked) or the rabbit albumin hydrogel is put in place, stitched to the inner wall of the pericardium and the opening is closed. Hemostasis is performed and the sternum, subcutaneous tissue and skin are closed. The cicatrix is disinfected and the animal woken. The animals are killed at 1-2-3-4-5-7-8 weeks, and the hydrogel/pericardium section removed for histological analysis. The histological analysis consists of tissue sections of interest fixed beforehand in a 10% neutral formaldehyde solution, and then stained using haematoxylin-eosin. After evaluation under the microscope, the extent of fibrosis and inflammation will be classified according to a scale of 0 where no fibrosis is present and 4 if granulomatous cells, cell proliferation, etc. are found.

This series of experiments has shown that after 8 weeks, the hydrogel is fully resorbed, that no inflammatory activity is found on the implant site and that only insignificant adhesion residues are observed here and there.

The preliminary results of the in vivo analysis carried out on rabbits therefore confirm the biocompatible and biodegradable properties, limiting the extent of post-surgical fibrosis. In fact, there has been no rejection reaction to implantation of the gel (biocompatibility), the material dissolved in 3 to 4 weeks (biodegradability) and finally the protection phenomenon limiting and even suppressing post-surgical fibrosis, the most important aspect, has been maintained so far.

### EXAMPLE 4

### Subcutaneous Implantation of a Hydrogel in Rabbits for the Controlled Release of Medication and Prevention of Post-Surgical Adhesions

Hydrogels with the same composition described previously have been synthesised with a final dimension of 12.5×10×1.5 mm. After being washed in a 0.9% NaCl physiological medium, the hydrogels are incubated for 3 hours either in a solution of 0.5% ibuprofen in the 0.9% NaCl, in a solution of 2% polyethylene glycol with a molecular mass of 4,000 Da or simply in a solution of 0.9% NaCl. The various hydrogels are then implanted subcutaneously in the dorsal position in the rabbit.

After 1-2-3-4-5-7-8 weeks, the rabbits were killed and histological sections were taken from the implant sites to be examined for adhesions, inflammation and hydrogel residues. The following observations were made, namely that the preliminary results confirm the bio compatible and biodegradable properties and a lower degree of post-surgical fibrosis in the presence of the active principles with which the hydrogels have been imbibed.

### EXAMPLE 5

### In Vitro Dissolution Rate of the Hydrogel

Hydrogels of varying compositions in terms of quantity of albumin and base were prepared and washed in a physiological buffer. They were incubated separately in a physiological buffer solution at 37° C. to assess the time needed for complete dissolution, i.e. to determine the time required for them to be reabsorbed by dissolution.

At 8 weeks, the gels were observed to have disintegrated, lost their initial consistency and mechanical strength; therefore, it is noted that biodegradation is effective in vitro after 8 weeks, which confirms the advantages of this invention.

1. A water-swellable, hydrophilic, physically crosslinked hydrogel which can slowly disintegrate in an aqueous medium, essentially consisting of albumin blood proteins which are bound in the form of a gel in a basic medium.

2. The hydrogel according to claim 1, wherein the albumin blood proteins are isolated from plasma or serum of human or animal origin.

3. The hydrogel according to claim 2, wherein the albumin of human origin comes from a blood bank, is autologous or obtained by genetic engineering.

4. A bioresorbable separation membrane comprising the hydrogel according to claim 1.

5. The bioresorbable separation membrane according to claim 4, wherein the hydrogel further comprises a therapeutically active substance.

6. The bioresorbable separation membrane according to claim 4, further comprising monomethoxy-poly(ethylene-glycol) chains on a surface that are linked by covalent bonds.

7. A veterinary or human surgical membrane comprising the bioresorbable separation membrane according to claim 4.

8. The bioresorbable separation membrane according to claim 5, wherein the therapeutically active substance is controllably released.

9. A process for the preparation of a hydrogel according to claim 1, which consists of mixing an aqueous solution of protein albumin with a base, then leaving the mixture to rest until polymerisation of the albumin monomolecules.

10. The process according to claim 9, wherein the aqueous solution of albumin is between 10 and 20% (weight/vol.), the base is NaOH 5N and the ratio between the NaOH and the albumin solution is between 0.5 and 2.0% (weight/vol.), and the duration of the polymerisation process lasts between about 1 h and 8 h.
